(19) United States
(12) Patent Application Publication
(10) Pub. No.: US 2025/0118402 A1
(43) Pub. Date: Apr. 10, 2025

(54) GENERATION METHOD AND GENERATION APPARATUS OF MEDICAL REPORT

(71) Applicant: Wistron Medical Technology Corporation, HSINCHU CITY (TW)

(72) Inventors: Han Chun Kuo, Hsinchu City (TW); Shih Feng Huang, Hsinchu City (TW); Chih Yi Chien, New Taipei City (TW); Chun Chun Tsai, New Taipei City (TW); Shao Wei Wu, New Taipei City (TW); Yu Fen Lin, New Taipei City (TW)

(73) Assignee: Wistron Medical Technology Corporation, HSINCHU CITY (TW)

(21) Appl. No.: 18/494,720

(22) Filed: Oct. 25, 2023

(30) Foreign Application Priority Data
Oct. 4, 2023 (TW) 112138142

(51) Int. Cl. G16H 15/00; G06N 20/00; G10L 15/06; G10L 15/19; G10L 15/22

(52) U.S. Cl. CPC G16H 15/00; G06N 20/00; G10L 15/063; G10L 15/19; G10L 15/22

(57) ABSTRACT

A generation method and generation apparatus of a medical report are provided. In the method, the writing style is analyzed from multiple historical texts, where the writing style includes multiple common words in the historical text and the contextual relationships that connect those common words; the medical data is converted into draft text that conforms to the template text, where the template text is a report that conforms to a preset style; and by using the draft text and writing style as input data of the language model, an output report that conforms to the writing style is generated, where the language model selects sentences that conform to the writing style.

### CROSS-REFERENCE TO RELATED APPLICATION

This application claims the priority benefit of Taiwan application serial no. 112138142 filed on Oct. 4, 2023. The entirety of the above-mentioned patent application is hereby incorporated by reference herein and made a part of this specification.

### BACKGROUND

### Technical Field

The disclosure relates to a machine learning technology, and in particular relates to a generation method and a generation apparatus of medical report by using machine learning technology.

### Description of Related Art

The generation of medical reports today typically relies on human effort, which may result in varying writing styles in the content of the medical reports. For instance, pathological reports may vary depending on the hospital, type of specimen, and the report writing habits of the physician.

### SUMMARY

A generation method and a generation apparatus of a medical report, which may automatically generate a customized report, are provided in the disclosure.

The generation method of a medical report according to the embodiment of the disclosure includes (but is not limited to) the following operation. A writing style is analyzed from multiple historical texts, where the writing style includes multiple common words in the historical texts and the contextual relationships that connect the common words. Medical data is converted into draft text that conforms to the template text, where the template text is a report that conforms to a preset style. An output report that conforms to the writing style is generated by using the draft text and writing style as input data of a language model, where the language model is configured to select sentences that conform to the writing style.

The generation apparatus of the medical report according to the embodiment of the disclosure includes (but is not limited to) a storage and a processor. The storage stores program code. The processor is coupled to the storage. The processor loads the program code and executes the following operation. A writing style is analyzed from multiple historical texts, where the writing style includes multiple common words in the historical texts and the contextual relationships that connect the common words. Medical data is converted into draft text that conforms to the template text, where the template text is a report that conforms to a preset style. An output report that conforms to the writing style is generated by using the draft text and writing style as input data of a language model, where the language model is configured to select sentences that conform to the writing style.

Based on the above, the generation method and generation apparatus of a medical report of the embodiment of the disclosure use medical data and various customized report templates, and automatically generate medical reports through the assistance and connection of a language model. In this way, customized reports may be provided and the accuracy and readability of the reports may be improved.

In order to make the above-mentioned features and advantages of the disclosure comprehensible, embodiments accompanied with drawings are described in detail below.

### BRIEF DESCRIPTION OF THE DRAWINGS

FIG. 1 is a block diagram of a generation apparatus according to an embodiment of the disclosure.

FIG. 2 is a flowchart of a generation method of a medical report according to an embodiment of the disclosure.

FIG. 3 is a flowchart of style training according to an embodiment of the disclosure.

FIG. 4 is a schematic diagram of a neural network configured for style training according to an embodiment of the disclosure.

FIG. 5 is a schematic diagram of a neural network configured for style training according to an embodiment of the disclosure.

FIG. 6 is a schematic diagram of pathological data according to an embodiment of the disclosure.

FIG. 7 is a flowchart of operation determination according to an embodiment of the disclosure.

FIG. 8 is a schematic diagram of the architecture of a language model according to an embodiment of the disclosure.

FIG. 9 is a schematic diagram of the training process of a language model according to an embodiment of the disclosure.

### DETAILED DESCRIPTION OF DISCLOSED EMBODIMENTS

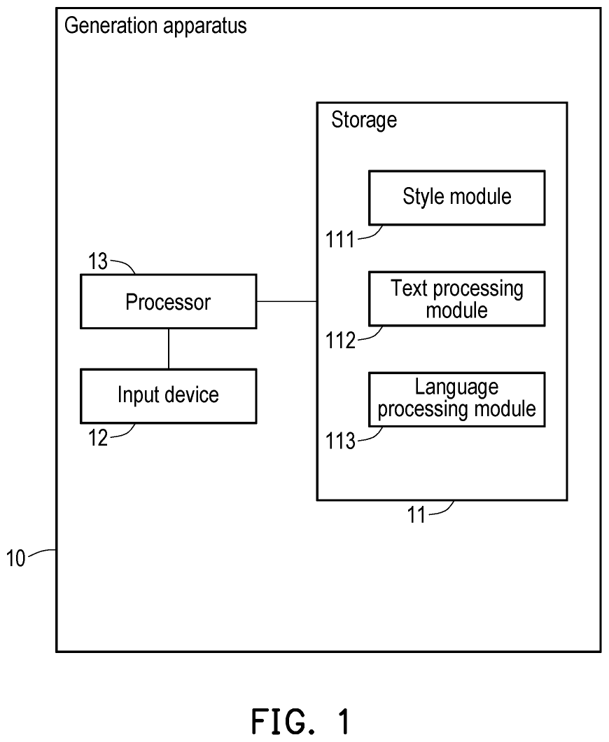

FIG. 1 is a block diagram of a generation apparatus 10 according to an embodiment of the disclosure. Referring to FIG. 1, the generation apparatus 10 includes (but is not limited to) a storage 11, an input device 12, and a processor 13. The generation apparatus 10 may be a computer host, a server, a smartphone, a tablet, a wearable device, a smart home appliance, a vehicle-mounted device, or other electronic devices.

The storage 11 may be any type of fixed or movable random access memory (RAM), read only memory (ROM), flash memory, conventional hard disk drive (HDD), solid-state drive (SSD) or similar components. In one embodiment, the storage 11 is configured to store program codes, software modules (e.g., a style module 111, a text processing module 112, and a language processing module 113), configurations, data (e.g., data, reports, or model parameters) or files, and the embodiments thereof are described in detail below.

The input device 12 may be a microphone, a keyboard, a mouse, a touch panel, or a transmission interface (e.g., USB, Lightning, or communication transceiver). In one embodiment, the input device 12 is configured to obtain medical data. Medical data may be specimen data, clinical data, medication records, operation records, test reports, consultation records, treatment records, emergency records, disease records and/or discharge/admission medical records. The content of the data may include identities, values, process records and/or diagnoses. For example, the user reads the content of the specimen data, the microphone receives the sound signal, and the sound signal may be converted into speech data (e.g., obtained through signal processing). For another example, the touch panel or keyboard receives input operations of surgical records. For another example, consultation records are obtained from a flash drive. However, there are many types and/or acquisition methods of medical data, and the embodiments of the disclosure are not limited thereto.

The processor 13 is coupled to the storage 11 and the communication transceiver 12. The processor 13 may be a central processing unit (CPU), a graphics processing unit (GPU), or other programmable general-purpose or special-purpose microprocessors, a digital signal processor (DSP), a programmable controller, a field programmable gate array (FPGA), an application-specific integrated circuit (ASIC), a neural network accelerator, or other similar components, or combinations of components thereof. In one embodiment, the processor 13 is configured to execute all or some of the operations of the generation apparatus 10, and may load and execute various program codes, software modules, files, and data stored in the storage 11. In some embodiments, the functions of the processor 13 may be realized by software or chips.

In one embodiment, the processor 13 executes the style module 111, the text processing module 112, and/or the language processing module 113. The functions of each module 111 to 113 are described in detail in subsequent embodiments.

Hereinafter, the method according to the embodiment of the disclosure is described in conjunction with various apparatuses, components, and modules in the generation apparatus 10. Each process of the method may be adjusted according to the implementation.

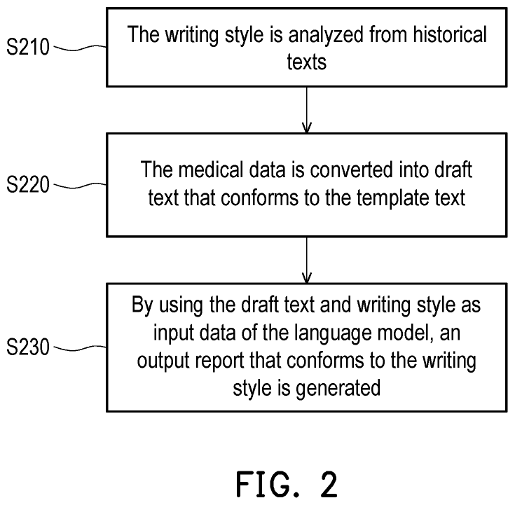

FIG. 2 is a flowchart of a generation method of a medical report according to an embodiment of the disclosure. Referring to FIG. 2, the processor 13 analyzes the writing style from multiple historical texts through the style module 111 (step S210). Specifically, the historical texts are written content that records one or more types of medical data (e.g., a pathological report, a consultation report, or a surgery report). Each historical text is originates from a previously written document, for example, the content of the historical text was input by a physician or examiner in the past through the input device 12 via voice, image, or input operation. Generally speaking, each individual has their own way of articulating written content, such as common words, connective terms between words, sentence segmentation methods, or word order within sentences. The processor 13 may define the writing style to include multiple common words in multiple historical texts and the contextual relationships that connect the common words. Common words may be words whose number of occurrences or proportions in multiple historical texts are higher than the corresponding threshold. Contextual relationships may include the order of adjacent words in the sentence, the content of the preceding and following words, the beginning and/or the end of the sentence. Contextual relationships are related to the arrangement of introduction, elucidation, transition and conclusion, or arrangement of bullet points in articles or sentences.

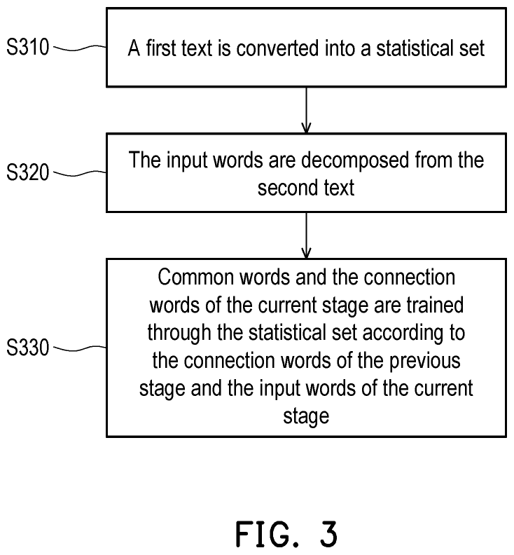

FIG. 3 is a flowchart of style training according to an embodiment of the disclosure. Referring to FIG. 1, the historical texts include multiple first texts and a second text. The processor 13 may select one of the historical texts as the second text, and use other historical texts as the first text. The processor 13 may convert the first texts into a statistical set through the style module 111 (step S310). Specifically, the statistical set includes the number of occurrences of multiple reference words in the first texts. The processor 13 may define the reference words. The reference words may be all or part of the words in the first text, preset words, or words defined based on user operations. The processor 13 respectively counts the number of occurrences of the reference words in each first text, and integrates these number of occurrences into a statistical set based on different word content and different texts.

In one embodiment, the statistical set is a parameter matrix. The processor 13 may use the number of occurrences of multiple reference words in the first texts as the values of multiple elements in the initial matrix through the style module 111. In the initial matrix, each row corresponds to a first text, and each column corresponds to a reference word. That is, each row is a vector representation of a piece of text.

For example, multiple pathological reports from a certain physician are as follows:
The content of the first article of the first texts is:
The specimen is formed of two pieces of gray-white soft tissue, measuring 0.1×0.1×0.1 cm. All specimens are embedded in one embedded cassette . . .
The content of the second article of the first texts is:
The specimen is formed of a piece of tissue, measuring 0.5×0.1×0.1 cm. All specimens are packaged in one embedded cassette . . .
Therefore, the initial matrix IM may be expressed as:

$$IM = [ \begin{matrix}2 , 2 , 1 , 2 , … , 1 \\ 2 , 2 , 1 , 1 , … , 1 \\ … \\ …\end{matrix} ] \qquad ( 1 )$$

The coordinate (2,1) represents 2, for instance, the term “specimen” appears 2 times in the second article of the first texts.

In one embodiment, the processor 13 may reduce the dimensions of the initial matrix to generate a parameter matrix. For example, the processor 13 performs singular value decomposition (SVD), principal component analysis (PCA) or other dimensionality reduction methods on the initial matrix. Some words with similar semantics are compressed to specific dimensions, allowing a matrix with a smaller dimension than the initial matrix to illustrate the semantic relationship between the first texts. The parameter matrix may be configured to predict multiple common words and connection words at the current stage, and are detailed in subsequent embodiments.

In another embodiment, the processor 13 may use the initial matrix as a parameter matrix or perform other conversions on the initial matrix.

Referring to FIG. 3, the processor 13 may decompose multiple input words from the second text through the style module 111 (step S320). Specifically, these input words are words that appear in the second text. In one embodiment, the processor 13 decomposes sentences through word embedding. Word embedding maps or embeds words in a text space into a numerical vector space. For example, the sentence is “the specimen is formed of three tissues”, which is broken down into “specimen”, “formed of”, “three”, and “tissue” through word embedding. Each word may correspond to a numerical value and may be represented by a binary vector. In other embodiments, the processor 13 may decompose the sentences in the second text through word comparison or other methods, and convert them into corresponding hyperparameters, numerical values, or vectors.

Referring to FIG. 3, the processor 13 may train one or more common words and the connection words of the current stage through the statistical set according to the connection words of the previous stage and the input words of the current stage through the style module 111 (step S330). Specifically, the style module 111 uses a neural network that processes sequence data. This neural network includes multiple stages, and the input of each stage includes the output of the previous stage and the input words of the second text. Multiple input words of the second text are respectively used as the input of one stage, and the order of the input words in the sentence corresponds to the order of multiple stages of the neural network. For example, the first input word in a sentence is input into the first stage, and the second input word in the sentence is input into the second stage, and so on. Each stage of the neural network outputs the common words and the connection words of the current stage. The “current” stage refers to the stage in which training is currently performed. Therefore, after one stage is completed (i.e., the common words and the connection words of this stage are output), the next stage is regarded as the current stage.

The contextual relationship of writing style includes the connection word between a previous word and a target word. The previous word precedes the target word in the sentence. The connecting words are suitable words that are between the previous word and the target word in the sentence, capable of connecting the previous word and the target word. By analogy, the connection words of the previous stage is predicted using the input words of the previous stage and the connection words of the stage before it, and the input words of the previous stage is positioned before the input words of the current stage in the second text.

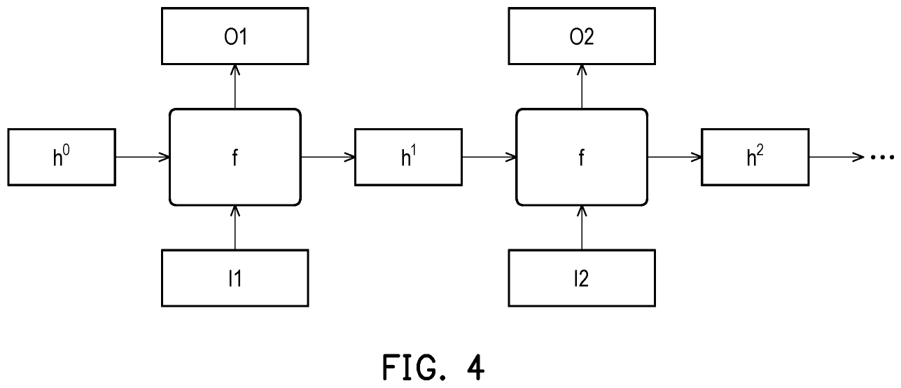

In one embodiment, one of the main concepts of the neural network is to perform dimensional mapping on the output of the previous stage and the input of the current stage through a linear function. For example, FIG. 4 is a schematic diagram of a neural network configured for style training according to an embodiment of the disclosure. Referring to FIG. 4, the linear function f in the first stage (located in the computing block on the left in the figure) performs dimensional mapping on the input word I1 (represented by hyperparameters, numerical values, or vectors to facilitate calculations) and the connection word h⁰, and generates the output O1 (including the connection word h¹). The connection word h⁰ is the numerical value representing the beginning of the sentence, for example, “0000”. By analogy, the linear function f in the next stage (located in the computing block on the right in the figure) performs dimensional mapping on the input word I2 (represented by hyperparameters, numerical values, or vectors to facilitate calculations) and the connection word h¹, and generates the output O2 (including the connection word h²). In addition, the common words in each stage may be generated according to the connection words of the corresponding stage, which is explained in subsequent embodiments.

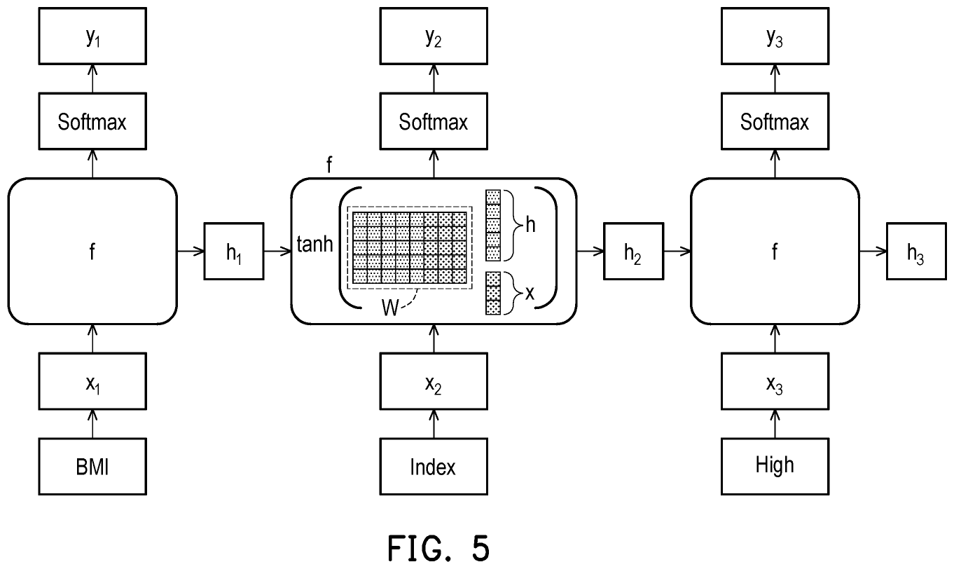

Regarding the linear function f, FIG. 5 is a schematic diagram of a neural network configured for style training according to an embodiment of the disclosure. Referring to FIG. 5, input words x₁ to x₃ (e.g., “BMI”, “index”, and “high”) are input to different stages of the neural network. The processor 13 may connect the connection words of the previous stage and the input words of the current stage into a vector to be predicted. Taking FIG. 5 as an example, in the previous stage, the connection word h (5×1 vector, such as the connection word h₁) and the input word x (3×1 vector, such as the input word x₂, in which the input words x₂ represents the “index” vector) is connected into an 8×1 vector.

The processor 13 may calculate the inner product of the parameter matrix W and the vector to be predicted. The parameter matrix W takes an 8×5 matrix as an example in FIG. 5, that is, the (compressed) number of occurrences of five articles of the first texts and eight reference words.

Next, the processor 13 generates the connection words of the current stage (e.g., the connection words h₂ in FIG. 5) by using the inner product of the parameter matrix W and the vector to be predicted as an input value of the activation function. The activation function may be a hyperbolic tangent (tanh) function, a softsign function, a rectified linear unit function (ReLU), or a transfer function. The activation function of each stage defines the output of this stage under a given input or set of inputs. That is to say, the inner product of the parameter matrix W and the vector to be predicted is calculated through an activation function to output the connection words.

In one embodiment, the processor 13 may select one from multiple connection words in the current stage as a common word in the current stage according to multiple occurrence probabilities corresponding to the connection words in the current stage. For example, the activation function may output the occurrence probabilities of multiple connection words (i.e., the occurrence probability with given preceding and following words), and select the one with the highest occurrence probability. The processor 13 may perform softmax on the connection words of the current stage. Softmax may compress a K-dimensional vector containing any real number into another K-dimensional real vector, so that each element ranges from 0 to 1, and the sum of all elements is 1. Taking FIG. 5 as an example, the processor 13 performs softmax on the connection words h₂ to output the common word y₂. The generation of other common words y₁ and y₃ may be deduced by analogy, and are not repeated herein. That is, the correlation between the common words of the contributors who wrote these historical texts has a higher probability or the highest probability of appearing together in the context.

In one embodiment, based on the cross validation mechanism, the processor 13 may select one of the multiple first texts of the current training program as the second text of another training program through the style module 111, and use the second text of the current training program as one of the first texts for the another training program. For example, FIG. 5 is a neural network configured for a certain training program. That is, multiple historical texts alternately serve as the input text for the neural network, the parameter matrix for calculations with the input words, or the statistical set configured for training in different training programs. For example, in the first training program, the first historical text serves as the second text, and the second and third historical texts serve as the first texts; in the second training program, the second historical text serves as the second text, and the first and third historical texts serve as the first texts. For multiple training programs, the processor 13 generates different parameter matrices (or statistical sets) and different input words of the second text.

Therefore, through the training of the above-mentioned neural network, and through a process of word chaining (that is, multiple stages of connection), the text input will more readily generate the common word of a particular contributor, thereby more closely approximating the writing style of this contributor.

Referring to FIG. 2, the processor 13 converts the medical data into draft text that conforms to the template text through the text processing module 112 (step S220). Specifically, this template text is a report that conforms to a preset style. For example, this specimen is formed of [quantity variable] tissues, measured as [length variable]×[width variable]×[height variable], and weighs [weight variable] grams. [Quantity variable], [length variable], [width variable], [height variable], and [weight variable] are variables in the template text. Different types of medical data have corresponding template texts. For example, different template texts are used for different specimens.

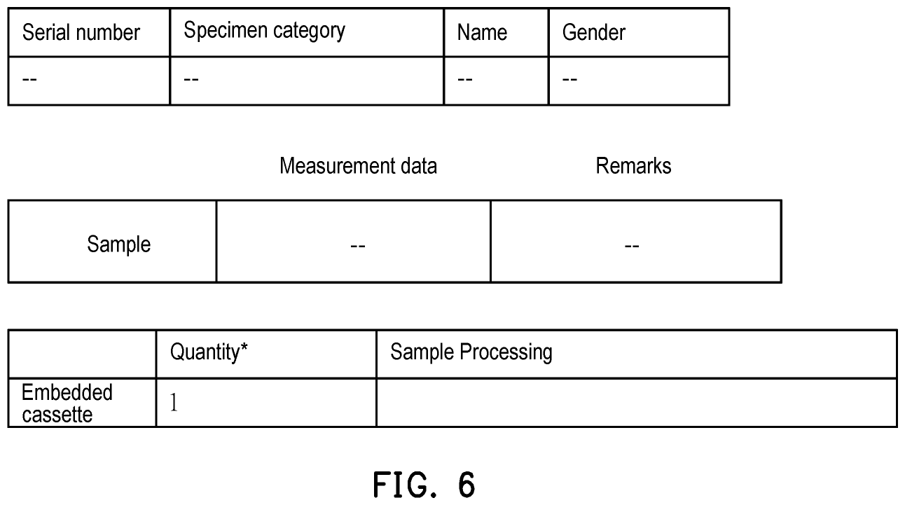

In one embodiment, the processor 13 may use the text processing module 112 to replace the variables in the template text with words or numerical values in the medical data. For example, FIG. 6 is a schematic diagram of pathological data according to an embodiment of the disclosure. Referring to FIG. 6, the pathological data records content such as serial number, specimen category, name, measurement data, remarks, number of packaging boxes, sample processing, etc. The length, width, and height in the measurement data can, for example, replace the [length variable], [width variable] and [height variable] in the template text. After replacing all or part of the variables, draft text may be generated by combining them with other word contents of the template text. In other words, compared to the template text, the variables are changed into words or numerical values of the medical data.

In one embodiment, the processor 13 may obtain voice data through the input device 12 and convert the voice data into medical data through speech-to-text conversion. Specifically, the microphone records sound and converts the sound signal into speech data. Next, the processor 13 recognizes the words of multiple medical data in the voice data through speech-to-text conversion, and associates the recognized words with the corresponding type of medical data. For example, the display (not shown) displays an image as shown in FIG. 6. Users may read out the contents of these medical data in sequence. The spoken content is segmented through specified input operations or specified terms. For example, for size, “times” is used as the segmentation word, and thereby 3.1 times 4.5 times 6 is segmented into “3.1”, “4.5”, and “6”. As another example, “less than” corresponds to the word “<”. For another example, “all specimens are packaged in one embedded cassette” corresponds to “the number of embedded cassettes is one”; “representative parts are embedded in two embedded cassettes” corresponds to “the number of embedded cassettes is greater than two”.

In one embodiment, the medical data may include measurement data of one or more specimens, and the measurement data of the specimens may include size (e.g., length, width, and/or height), quantity, and/or weight, for example, the content recorded in the pathological data shown in FIG. 6. However, medical data is not limited to specimen data, for example, it may be the content of the consultation report, the process of the surgical report, or the medication process.

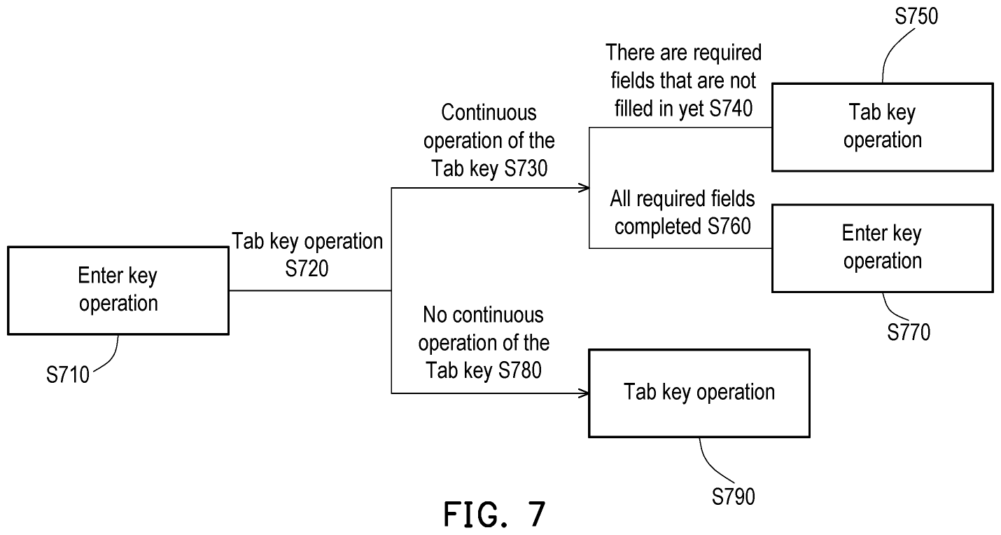

FIG. 7 is a flowchart of operation determination according to an embodiment of the disclosure. Referring to FIG. 7, keys in a keyboard (i.e., the input device) may be configured to assist in inputting medical data. Taking the Enter and Tab keys as an example, the processor 13 receives the Enter key operation through the input device 12 (step S710). After receiving the Tab key operation (step S720), if there is a continuous operation of the Tab key (step S730), the “save” operation is triggered. For example, receiving two consecutive Tab key operations within one second may trigger a “save” operation. Next, the processor 13 confirms whether there are required fields that are not filled in yet. These fields appear, for example, in various items in FIG. 6. If there are required fields that are not filled in yet (step S740), the Tab key operation is triggered (step S750), for example, the next required field that is not filled in yet is searched until the last unfilled required field, and finally the Enter key operation is triggered. If all required fields are completed (step S760), the Enter key operation is triggered and the pathological data is stored accordingly (step S770). On the other hand, if the continuous operation of the Tab key is not received (step S780), the Tab key operation is triggered (step S790).

In other embodiments, medical data may be input through image recognition words or input operations. For example, the processor 13 recognizes words in the pathological report through image recognition technology, and respectively associates the recognized words with the corresponding type of medical data. For another example, the processor 13 directly receives numerical values or word inputs through a keyboard.

Referring to FIG. 2, the processor 13 generates an output report that conforms to the writing style by using the language processing module 113 and using the draft text and writing style as input data of the language model (step S230). Specifically, the output report is the output data of the language model, and the language model is configured to select sentences that conform to the writing style of a certain contributor, for example, the contributor to the aforementioned historical text. The language model may be a large language model (LLM) or other neural language models (NLM). A language model is a machine learning model configured to understand and generate human language, for example, it may be GPT, LLAMA, or LaMDA. Language models may produce reports that conform to the writing style of a specific contributor. During the generation process of the output report, the processor 13 may store the sentences selected by the language model into a temporary storage region, so that sentences that conform to the specific writing style may be subsequently selected for replacement or addition. While adding the sentences selected by the language model to the temporary storage region, the processor 13 may perform keyword analysis for use by the neural network employed by the text processing module 112.

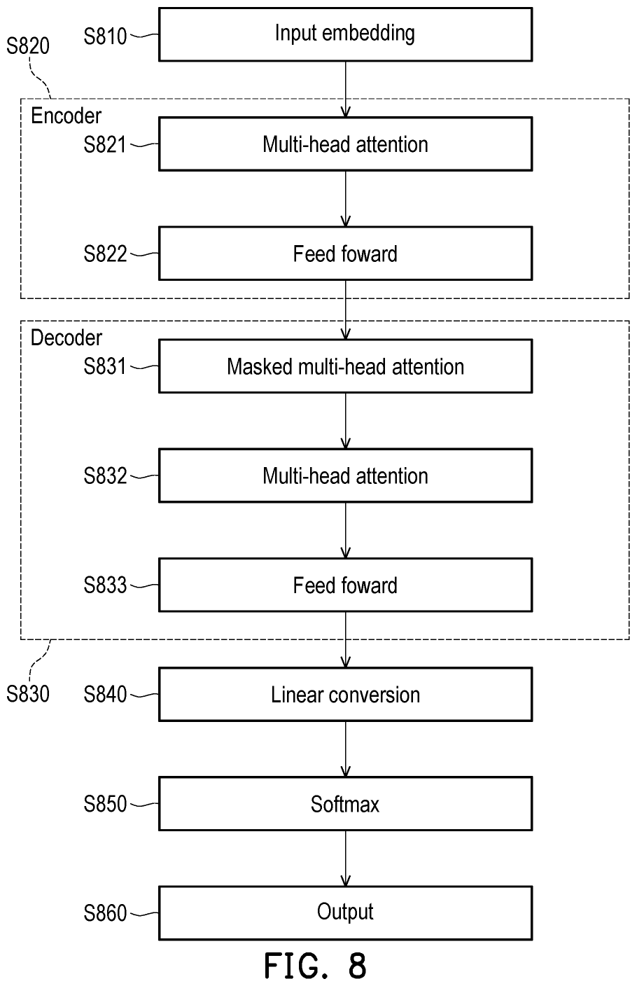

In one embodiment, the language model is a transformer architecture, and the transformer architecture includes an encoder and a decoder. For example, FIG. 8 is a schematic diagram of the architecture of a language model according to an embodiment of the disclosure. Referring to FIG. 8, the processor 13 performs input embedding to the writing style (e.g., common words and connection words) and the draft text generated in step S210 through the language processing module 113 (step S810). For example, sentences are decomposed through word embedding and the numerical value of the decomposed word is generated. In the encoder (step S820), the processor 13 provides multi-head attention (step S821) and feed forward (step S822). Multi-head attention may simultaneously focus on information at different positions when processing the input sequence, thereby capturing the correlation and dependence in the input sequence. In addition, feed forward networks may be formed of one or more linear transformations and nonlinear activation functions. The input of the feed forward network is a word vector, and after being processed by a series of linear transformations and activation functions, another word vector may be output.

The processor 13 takes the output of the encoder as input to the decoder. In the decoder (step S830), the processor 13 provides masked multi-head attention (step S831), multi-head attention (step S832) and feed forward (step S833). One of the purposes of masking technology is to focus on the input sequence during decoding to avoid information leakage and interference with future data. For example, a padding mask sets part of the attention weight to a smaller numerical value than other numerical values, or a sequence mask sets the attention weight of a certain position in the future to a smaller numerical value than other numerical values. For description of multi-head attention (step S832) and feed forward (step S833), reference may be respectively made to the description of step S821 and step S822, and are not repeated herein. Through multi-head attention, each head may focus on different attention positions, and the information from these positions may be merged together in the final output. In this transformer architecture, a multi-head attention mechanism may be provided through the encoder and decoder respectively.

Next, the processor 13 linearly converts the output of the decoder (step S840), and generates an output (step S860) through softmax (step S850). One of the properties of language models is the production of natural and fluent text, for example, generating coherent and logical text content such as articles, stories, questions and answers according to given context or prompts. In other words, the language model may re-edit the draft text, select suitable sentences according to common words and connection words provided by the writing style, and combine them into an output report. Depending on the application requirements, the output report may be a pathological report, a consultation report, a health examination report, a surgery report, or other medical or clinical-related reports.

It should be noted that the architecture shown in FIG. 8 is only used as an example, and other architectures may also be used in other embodiments.

In one embodiment, the processor 13 may use a large amount of pre-training data to learn the language model. In the pre-training stage, the language model learns the relationship between words and sentences through large-scale text data sets to gain understanding and reasoning capabilities of language structure.

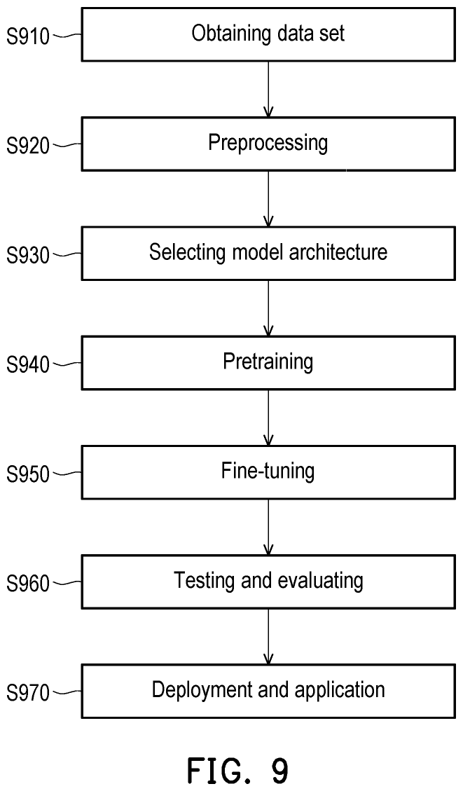

For example, FIG. 9 is a schematic diagram of the training process of a language model according to an embodiment of the disclosure. Referring to FIG. 9, in step S910, the processor 13 may obtain a data set for training by using the public data set as a text data set. Public data may be text from web pages, books, news articles, or other sources.

In step S920, the processor 13 may pre-process the data of the data set. For example, text is split into sequences of words or subwords through tokenization, and these words or subwords are encoded for subsequent model training.

In step S930, the processor 13 may select a model architecture (step S930). For example, the transformer architecture shown in FIG. 8. This transformer architecture has multiple layers of self-attention and feed forward neural networks. Before training, the processor 13 may define hyperparameters such as the depth of the model, the number of attention heads, and the dimension of the hidden layer. However, the architecture of the language model in the embodiment of the disclosure is not limited to the transformer architecture.

In step S940, the processor 13 learns the parameters in the language model through pre-training. This stage may be divided into two steps: self-supervised pre-training and supervised fine-tuning. In self-supervised pre-training, unlabeled text data may be used, such as text obtained from the Internet. The model learns to predict the context of words by adopting self-predictive tasks, thereby gaining an understanding of the language structure. In supervised fine-tuning, after self-supervised pre-training, labeled data, such as question-answer pairs, translation pairs, etc., are used to fine-tune the language model and make it more suitable for a specific task. This step contributes to improving the performance of the model on a specific task.

In step S950, after the pre-training stage, the processor 13 may further fine-tune the language pre-training model to adapt to the specific application field. For example, a model is trained on a textual data set of physician health examination reports to adjust the parameters of the model to improve performance.

In step S960, after the model fine-tuning, the processor 13 may perform testing and evaluation to evaluate the performance of the language model on different tasks. For example, language models are tested using test data sets and various evaluation metrics are calculated. For example, accuracy and physician feedback (human feedback) are used to measure the performance of the model.

In step S970, after completing the training and evaluation, the processor 13 may deploy the language model into actual applications, for example, it is used in step S230. In some application scenarios, the language model training process may require more computing resources and time, and may be performed on a large-scale computing cluster. The training process requires multiple iterations and adjustments to achieve optimal performance. In addition, the language model may be updated and maintained regularly or irregularly to ensure its effectiveness and efficiency in the face of changing text data and tasks.

To sum up, in the generation method and generation apparatus of a medical report of the according to the embodiments of the embodiment of the disclosure, a writing style (e.g., common words and the contextual relationships of these common words) may be learned from historical texts through a neural network, and current medical data may be converted into a draft text in a preset style. Then, an output report is generated according to the writing style and draft text by using the language model. This output report is a report that records medical data and conforms to a specific writing style. In addition, medical data may be recorded via voice.

In an application scenario, corresponding template texts are selected for different specimens, and then the specimen data is input using voice input. When a physician or medical staff wants to read a report, the generation apparatus may generate a report corresponding to the style or form of the physician or medical staff through a previously pre-trained language model. When the physician or medical staff modifies or adjusts the output report, the adjustment may be fed back to the language model for further reinforcement training. Even if the physician or medical staff does not modify or adjust the output report, it may be used as another kind of feedback for further reinforcement training of the language model. Embodiments of the disclosure may provide individual reinforcement training based on feedback from individual users, which is different from the training of existing language models.

In this way, medical reports may be automatically generated for the user end and the accuracy and readability of the reports may be improved. Physicians only need to check and modify the report content, thereby increasing the speed of report completion and reducing the waiting time for patients to receive reports. In addition, it may reduce the time physicians spend writing reports, allowing them to focus more on the diagnosis and research of lesions.

Although the disclosure has been described in detail with reference to the above embodiments, they are not intended to limit the disclosure. Those skilled in the art should understand that it is possible to make changes and modifications without departing from the spirit and scope of the disclosure. Therefore, the protection scope of the disclosure shall be defined by the following claims.

What is claimed is:

1. A generation method of a medical report, comprising:
analyzing a writing style from a plurality of historical texts, the writing style comprising a plurality of common words in the historical texts and a contextual relationship that connects the common words;
converting medical data into a draft text that conforms to a template text, wherein the template text is a report that conforms to a preset style; and
generating an output report that conforms to the writing style by using the draft text and the writing style as input data of a language model, wherein the language model selects sentences that conform to the writing style.

2. The generation method of the medical report according to claim 1, wherein the historical texts comprise a plurality of first texts and a second text, the contextual relationship comprises a connection word between a previous word and a target word, the previous word precedes the target word in a sentence, and converting the medical data into the draft text that conforms to the template text comprises:
converting the first texts into a statistical set, wherein the statistical set comprises a number of occurrences of a plurality of reference words;
decomposing a plurality of input words from the second text, wherein the input words appear in the second text; and
training one of the common words and a connection word of a current stage through the statistical set according to a connection word of a previous stage and an input word of the current stage, wherein the connection word of the previous stage is predicted using an input word of the previous stage, and the input word of the previous stage is positioned before the input word of the current stage in the second text.

3. The generation method of the medical report according to claim 2, wherein the statistical set is a parameter matrix, and converting the first texts into the statistical set comprises:
using the number of occurrences of the reference words in the first texts as values of a plurality of elements in an initial matrix; and
reducing a dimension of the initial matrix to generate the parameter matrix, wherein the parameter matrix is configured to predict the common words and the connection word at the current stage.

4. The generation method of the medical report according to claim 3, wherein training one of the common words and the connection word of the current stage through the statistical set according to the connection word of the previous stage and the input word of the current stage comprises:
connecting the connection word of the previous stage and the input word of the current stage into a vector to be predicted;
calculating an inner product of the parameter matrix and the vector to be predicted;
generating the connection word of the current stage by using the inner product as an input value of an activation function.

5. The generation method of the medical report according to claim 4, wherein generating the connection word of the current stage comprises:
selecting one from a plurality of connection words in the current stage as one of the common word according to a plurality of occurrence probabilities corresponding to a plurality of connection words in the current stage.

6. The generation method of the medical report according to claim 5, wherein
the activation function is a hyperbolic tangent (tanh) function, a softsign function, a rectified linear unit function (ReLU), or a transfer function; or
reducing the dimension of the initial matrix comprises:
performing a singular value decomposition (SVD), a principal component analysis (PCA), or dimensionality reduction on the initial matrix; or
selecting one from the connection words in the current stage as one of the common words according to the occurrence probabilities of the connection words in the current stage comprises:
performing softmax on the connection words of the current stage.

7. The generation method of the medical report according to claim 2, further comprising:
selecting one of the first texts as a second text of another training program; and
using the second text as one of the first texts for the another training program.

8. The generation method of the medical report according to claim 1, wherein the language model is a transformer architecture, the transformer architecture comprises an encoder and a decoder, and generating the output report that conforms to the writing style comprises:
providing a multi-head attention mechanism through the encoder and the decoder respectively; and
taking an output of the encoder as an input to the decoder.

9. The generation method of the medical report according to claim 1, wherein converting the medical data into the draft text that conforms to the template text comprises:
replacing variables in the template text with words or numerical values in the medical data.

10. The generation method of the medical report according to claim 1, further comprising:
converting voice data into the medical data through speech-to-text conversion, wherein the medical data comprises measurement data of at least one specimen, and the measurement data of the at least one specimen comprises size, quantity, and/or weight.

11. A generation apparatus of a medical report, comprising:
a storage, stores a program code; and
a processor, coupled to the storage, loading the program code and executing:
analyzing a writing style from a plurality of historical texts, the writing style comprising a plurality of common words in the historical texts and a contextual relationship that connects the common words;
converting medical data into a draft text that conforms to a template text, wherein the template text is a report that conforms to a preset style; and
generating an output report that conforms to the writing style by using the draft text and the writing style as input data of a language model, wherein the language model selects sentences that conform to the writing style.

12. The generation apparatus of the medical report according to claim 11, wherein the historical texts comprise a plurality of first texts and a second text, the contextual relationship comprises a connection word between a previous word and a target word, the previous word precedes the target word in a sentence, and the processor further executes:
converting the first texts into a statistical set, wherein the statistical set comprises a number of occurrences of a plurality of reference words;
decomposing a plurality of input words from the second text, wherein the input words appear in the second text; and
training one of the common words and a connection word of a current stage through the statistical set according to a connection word of a previous stage and an input word of the current stage, wherein the connection word of the previous stage is predicted using an input word of the previous stage, and the input word of the previous stage is positioned before the input word of the current stage in the second text.

13. The generation apparatus of the medical report according to claim 12, wherein the statistical set is a parameter matrix, and the processor further executes:
using the number of occurrences of the reference words in the first texts as values of a plurality of elements in an initial matrix; and
reducing a dimension of the initial matrix to generate the parameter matrix, wherein the parameter matrix is configured to predict the common words and the connection word at the current stage.

14. The generation apparatus of the medical report according to claim 13, wherein the processor further executes:
connecting the connection word of the previous stage and the input word of the current stage into a vector to be predicted;
calculating an inner product of the parameter matrix and the vector to be predicted;
generating the connection word of the current stage by using the inner product as an input value of an activation function.

15. The generation apparatus of the medical report according to claim 14, wherein the processor further executes:
selecting one from a plurality of connection words in the current stage as one of the common words according to a plurality of occurrence probabilities corresponding to a plurality of connection words in the current stage.

16. The generation apparatus of the medical report according to claim 15, wherein
the activation function is a hyperbolic tangent function, a softsign function, a rectified linear unit function, or a transfer function; or
the processor further executes:
performing a singular value decomposition, a principal component analysis, or dimensionality reduction on the initial matrix; or
performing softmax on the connection words of the current stage.

17. The generation apparatus of the medical report according to claim 12, wherein the processor further executes:
selecting one of the first texts as a second text of another training program; and
using the second text as one of the first texts for the another training program.

18. The generation apparatus of the medical report according to claim 11, wherein the language model is a transformer architecture, the transformer architecture comprises an encoder and a decoder, and the processor further executes:
providing a multi-head attention mechanism through the encoder and the decoder respectively; and
taking an output of the encoder as an input to the decoder.

19. The generation apparatus of the medical report according to claim 11, wherein the processor further executes:
replacing variables in the template text with words or numerical values in the medical data.

20. The generation apparatus of the medical report according to claim 11, wherein the processor further executes:
converting voice data into the medical data through speech-to-text conversion, wherein the medical data comprises measurement data of at least one specimen, and the measurement data of the at least one specimen comprises size, quantity, and/or weight.
